(19) United States
(12) Patent Application Publication
(10) Pub. No.: US 2023/0069182 A1
(43) Pub. Date: Mar. 2, 2023

(54) METHODS, SYSTEMS, AND COMPUTER PROGRAM PRODUCTS FOR AUTOMATICALLY ENRICHING A CLAIM WITH ADDITIONAL CLINICAL INFORMATION

(71) Applicant: Change Healthcare Holdings LLC, Nashville, TN (US)

(72) Inventors: Mike Peresie, Milton, GA (US); Ed Hafner, Boerne, TX (US)

(21) Appl. No.: 17/412,444

(22) Filed: Aug. 26, 2021

(51) Int. Cl. G06Q 40/08; G16H 10/60; G16H 40/20; G16H 70/20; G16H 50/20; G06F 16/245

(52) U.S. Cl. CPC G06Q 40/08; G16H 10/60; G16H 40/20; G16H 70/20; G16H 50/20; G06F 16/245

(57) ABSTRACT

A method includes receiving a claim associated with a patient from a provider; automatically determining whether additional clinical information is needed to adjudicate the claim; automatically retrieving the additional clinical information responsive to determining that the additional clinical information is needed to adjudicate the claim; communicating the claim and the additional clinical information to a payor responsive to determining that the additional clinical information is needed to adjudicate the claim; and communicating the claim to the payor responsive to determining that the additional clinical information is not needed to adjudicate the claim.

### FIELD

The present inventive concepts relate generally to health care systems and services and, more particularly, to processing of claims generated by providers for payment by payors.

### BACKGROUND

Health care service providers have patients that pay for their care using a variety of different payors. For example, a medical facility or practice may serve patients that pay by way of different insurance companies including, but not limited to, private insurance plans, government insurance plans, such as Medicare, Medicaid, and state or federal public employee insurance plans, and/or hybrid insurance plans, such as those that are sold through the Affordable Care Act. When providers submit claims to the payors for payment, the claims can be denied in whole or in part for a variety of different reasons. One such reason is that during adjudication of the claim the payor determines that before a final determination can be made whether to approve the claim for payment, additional clinical information is needed that doesn't currently accompany the claim. Retrieving additional clinical information to supplement a claim is typically a manual process. For example, a payor may be required to contact a provider and make arrangements for the provider to communicate a patient's medical record to the payor, which may be in electronic or paper form. The payor may then manually process the record that is received from the provider to associate all of the record or a portion of the record with the claim for further processing. This manual process to supplement the claim with additional clinical information may be time consuming and expensive for the payor.

### SUMMARY

According to some embodiments of the inventive concept, a method comprises: receiving a claim associated with a patient from a provider; automatically determining whether additional clinical information is needed to adjudicate the claim; automatically retrieving the additional clinical information responsive to determining that the additional clinical information is needed to adjudicate the claim; communicating the claim and the additional clinical information to a payor responsive to determining that the additional clinical information is needed to adjudicate the claim; and communicating the claim to the payor responsive to determining that the additional clinical information is not needed to adjudicate the claim.

In other embodiments, determining whether the additional clinical information is needed to adjudicate the claim comprises: determining that additional clinical information is needed to adjudicate the claim when the claim includes one or more procedure codes known to require the additional clinical information, the claim includes one or more procedure codes indicative of a health care procedure of questionable medical necessity, the claim includes information of questionable accuracy, or the claim includes one or more procedure codes that are inconsistent with a risk adjustment factor associated with the patient.

In still other embodiments, automatically retrieving the additional clinical information comprises accessing a directory that includes a plurality of retrieval techniques for retrieving the additional clinical information that are ranked in order of priority; and applying respective ones of the plurality of retrieval techniques in the order of priority to retrieve the additional clinical information.

In still other embodiments, the method further comprises: receiving the plurality of retrieval techniques that are ranked in order of priority from the provider; and storing the plurality of retrieval techniques in the directory.

In still other embodiments, the plurality retrieval techniques comprises an automated data pull technique and a request-response technique.

In still other embodiments, the plurality of retrieval techniques comprises accessing a storage location to retrieve the additional clinical information using Fast Healthcare Interoperability Resources (FHIR) protocol, sending a request to the provider to return the additional clinical information in an Electronic Data Interchange (EDI) format, or sending an electronic mail message to the provider requesting the additional clinical information.

In still other embodiments, automatically determining whether additional clinical information is needed to adjudicate the claim comprises: generating a claim completeness model based on adjudication of historical claims in which associations are determined between procedure codes in the historical claims and a need for additional clinical information.

In still other embodiments, generating the claim completeness model comprises: using an Artificial Intelligence (AI) system to learn the associations between the procedure codes in the historical claims and the need for additional clinical information.

In still other embodiments, automatically retrieving the additional clinical information comprises: retrieving a medical record associated with the patient; and processing the medical record to extract the additional clinical information therefrom.

In still other embodiments, processing the medical record to extract the additional clinical information therefrom comprises: generating a clinical information enrichment model based on supplementation of historical claims with patient medical record information.

In still other embodiments, generating the clinical information enrichment model comprises: using an Artificial Intelligence (AI) system to learn associations between procedure codes in the historical claims and the patient medical record information.

In some embodiments of the inventive concept, a system comprises a processor and a memory coupled to the processor and comprising computer readable program code embodied in the memory that is executable by the processor to perform operations comprising: receiving a claim associated with a patient from a provider; automatically determining whether additional clinical information is needed to adjudicate the claim; automatically retrieving the additional clinical information responsive to determining that the additional clinical information is needed to adjudicate the claim; communicating the claim and the additional clinical information to a payor responsive to determining that the additional clinical information is needed to adjudicate the claim; and communicating the claim to the payor responsive to determining that the additional clinical information is not needed to adjudicate the claim.

In further embodiments, determining whether the additional clinical information is needed to adjudicate the claim comprises: determining that additional clinical information is needed to adjudicate the claim when the claim includes one or more procedure codes known to require the additional clinical information, the claim includes one or more procedure codes indicative of a health care procedure of questionable medical necessity, the claim includes information of questionable accuracy, or the claim includes one or more procedure codes that are inconsistent with a risk adjustment factor associated with the patient.

In still further embodiments, automatically retrieving the additional clinical information comprises: accessing a directory that includes a plurality of retrieval techniques for retrieving the additional clinical information that are ranked in order of priority; and applying respective ones of the plurality of retrieval techniques in the order of priority to retrieve the additional clinical information.

In still further embodiments, automatically determining whether additional clinical information is needed to adjudicate the claim comprises: generating a claim completeness model based on adjudication of historical claims in which associations are determined between procedure codes, provider names, and/or patient risk adjustment factors in the historical claims and a need for additional clinical information.

In still further embodiments, generating the claim completeness model comprises: using an Artificial Intelligence (AI) system to learn the associations between the procedure codes, the provider names, and/or the patient risk adjustment factors in the historical claims and the need for additional clinical information.

In still further embodiments, automatically retrieving the additional clinical information comprises: retrieving a medical record associated with the patient; and processing the medical record to extract the additional clinical information therefrom.

In still further embodiments, processing the medical record to extract the additional clinical information therefrom comprises: generating a clinical information enrichment model based on supplementation of historical claims with patient medical record information.

In still further embodiments, generating the clinical information enrichment model comprises: using an Artificial Intelligence (AI) system to learn associations between procedure codes, provider names, and/or patient risk adjustment factors in the historical claims and the patient medical record information.

In some embodiments of the inventive concept, a computer program product comprises a non-transitory computer readable storage medium comprising computer readable program code embodied in the medium that is executable by a processor to perform operations comprising: receiving a claim associated with a patient from a provider; automatically determining whether additional clinical information is needed to adjudicate the claim; automatically retrieving the additional clinical information responsive to determining that the additional clinical information is needed to adjudicate the claim; communicating the claim and the additional clinical information to a payor responsive to determining that the additional clinical information is needed to adjudicate the claim; and communicating the claim to the payor responsive to determining that the additional clinical information is not needed to adjudicate the claim.

It is noted that aspects described with respect to one embodiment may be incorporated in different embodiments although not specifically described relative thereto. That is, all embodiments and/or features of any embodiments can be combined in any way and/or combination. Moreover, other methods, systems, articles of manufacture, and/or computer program products according to embodiments of the inventive concept will be or become apparent to one with skill in the art upon review of the following drawings and detailed description. It is intended that all such additional systems, methods, articles of manufacture, and/or computer program products be included within this description, be within the scope of the present inventive subject matter and be protected by the accompanying claims.

### BRIEF DESCRIPTION OF THE DRAWINGS

Other features of embodiments will be more readily understood from the following detailed description of specific embodiments thereof when read in conjunction with the accompanying drawings, in which:

FIG. 1 is a block diagram that illustrates a communication network including a claim enrichment system for supplementing a claim with additional clinical information in accordance with some embodiments of the inventive concept;

FIG. 2 is a flowchart that illustrates operations for supplementing a claim with additional clinical information in accordance with some embodiments of the inventive concept;

FIGS. 3 and 4 are block diagrams that illustrate the claim enrichment system in accordance with some embodiments of the inventive concept;

FIGS. 5 and 6 are flowcharts that illustrate operations for supplementing a claim with additional clinical information in accordance with further embodiments of the inventive concept;

FIG. 7 is a block diagram that illustrates the claim enrichment system in accordance with further embodiments of the inventive concept;

FIG. 8 is a data processing system that may be used to implement a claim enrichment system for supplementing a claim with additional clinical information in accordance with some embodiments of the inventive concept; and

FIG. 9 is a block diagram that illustrates a software/hardware architecture for use in in a claim enrichment system for supplementing a claim with additional clinical information in accordance with some embodiments of the inventive concept.

### DETAILED DESCRIPTION

In the following detailed description, numerous specific details are set forth to provide a thorough understanding of embodiments of the inventive concept. However, it will be understood by those skilled in the art that embodiments of the inventive concept may be practiced without these specific details. In some instances, well-known methods, procedures, components, and circuits have not been described in detail so as not to obscure the inventive concept. It is intended that all embodiments disclosed herein can be implemented separately or combined in any way and/or combination. Aspects described with respect to one embodiment may be incorporated in different embodiments although not specifically described relative thereto. That is, all embodiments and/or features of any embodiments can be combined in any way and/or combination.

As used herein, the term “provider” may mean any person or entity involved in providing health care products and/or services to a patient.

Embodiments of the inventive concept are described herein in the context of a claim enrichment system for supplementing a claim with additional clinical information that includes an artificial intelligence (AI) engine, which uses machine learning. It will be understood that embodiments of the inventive concept are not limited to a machine learning implementation of the claim enrichment system and other types of AI systems may be used including, but not limited to, a multi-layer neural network, a deep learning system, a natural language processing system, and/or computer vision system. Moreover, it will be understood that the multi-layer neural network is a multi-layer artificial neural network comprising artificial neurons or nodes and does not include a biological neural network comprising real biological neurons.

Some embodiments of the inventive concept stem from a realization that the use of an intermediary located in the cloud, such as a clearinghouse for processing claims generated by providers, may be configured to determine which claims are likely to need additional clinical information before a payor can fully adjudicate the claim and approve the claim for payment. This may alleviate the payor from the manual and potentially time consuming and expensive burden of retrieving clinical information from a provider to ensure that a claim has sufficient information to allow the payor to adjudicate the claim for payment. For example, in some embodiments, a claim enrichment system may be configured to automatically process a claim destined for a payor to make a determination whether additional clinical information is needed to adjudicate the claim and that this additional clinical information has not already been included as a supplement or addendum to the claim. Various factors may be used to make this determination including the presence of one or more procedure codes (e.g., Current Procedural Terminology (CPT) codes) that are indicative of diagnoses, procedures, services, medications, or other forms of health care services or products that may require additional clinical information from the patient's medical record before a payor can authorize payment. These procedure codes may be indicative of services and/or products that are of questionable medical necessity. Another factor that may be considered is whether the claim includes information of questionable accuracy. The claim enrichment system may be configured to analyze the claim for internal consistency and may consider the particular provider that is submitting the claim. For example, some providers may develop a reputation for submitting claims with errant or incorrect information. Thus, the claim enrichment system may be configured to identify claims submitted from certain providers as requiring additional clinical information to support the services and/or medications provided that are listed thereon. Yet another factor that may be considered is whether the claim has one or more procedure codes that are inconsistent with a risk adjustment factor associated with the patient. Risk adjustment is a methodology that equates the health status of a patient to a number, called a risk score, to predict healthcare costs. The “risk” to a health plan insurer with expected high healthcare use is “adjusted” by also insuring members with anticipated lower healthcare costs. Risk adjustment is how payors participating in specific programs get payment for managing the healthcare needs of members based on their diagnoses. Thus, if a provider submits a claim for a patient with services and/or products that are inconsistent with the expected healthcare use for that patient based on the patient's risk adjustment factor, then additional clinical information from the patient's record may be required to support these services and/or products before a payor authorizes payment for the claim. In accordance with various embodiments of the inventive concept, the claim enrichment system may use a variety of techniques for analyzing the claims to determine whether additional clinical information is needed to adjudicate the claim including, for example, applying business rules received from a payor to the claims and/or generating a claim completeness model by using an Artificial Intelligence (AI) system to learn associations between procedure codes and/or other information (e.g., provider name, patient risk adjustment factors, and the like) contained in historical claims and the need for additional clinical information. The AI generated claim completeness model may then be used to process the claims received from one or more providers destined for a payor.

When a determination is made that additional clinical information is needed, the claim enrichment system may be configured to automatically retrieve the additional clinical information needed to supplement the claim without the need for manual intervention. In some embodiments, a directory may be maintained in which one or more retrieval techniques are stored in priority order. These retrieval techniques and rankings may be provided by a provider and stored in the directory and may include, but are not limited to, accessing a storage location to retrieve the additional clinical information using Fast Healthcare Interoperability Resources (FHIR) protocol, sending a request to the provider to return the additional clinical information in an Electronic Data Interchange (EDI) format, or sending an electronic mail message to the provider requesting the additional clinical information. The storage area may be implemented in a variety of different ways in accordance with various embodiments of the inventive concept. For example, the storage area may be part of one or more clinical networks that may be used to implement a local area, wide area, or national health information exchange. In other embodiments, a provider may implement its own network endpoint for housing its own clinical data. The provider may grant other entities direct access to its own electronic health records stored at this end point.

Once additional clinical information is retrieved by, for example, retrieving a patient's medical record, the additional clinical information may be communicated along with the claim to the payor. A patient's medical record may be large and include much information that is not needed by the payor to adjudicate the claim. In some embodiments, the claim enrichment system may be configured to process the medical record to extract a portion of the patient's medical record that is relevant to adjudication of the claim. In some embodiments, the relevant portion of the medical record may be extracted by generating a clinical information enrichment model that uses an AI system to learn associations between procedure codes and/or other information (e.g., provider name, patient risk adjustment factors, and the like) contained in historical claims and patient medical record information.

Thus, a claim enrichment system according to some embodiments of the inventive concept may improve the efficiency and thereby reduce costs for payors when adjudicating claims from providers by eliminating manual tasks performed to obtain clinical information from providers to supplement the claims.

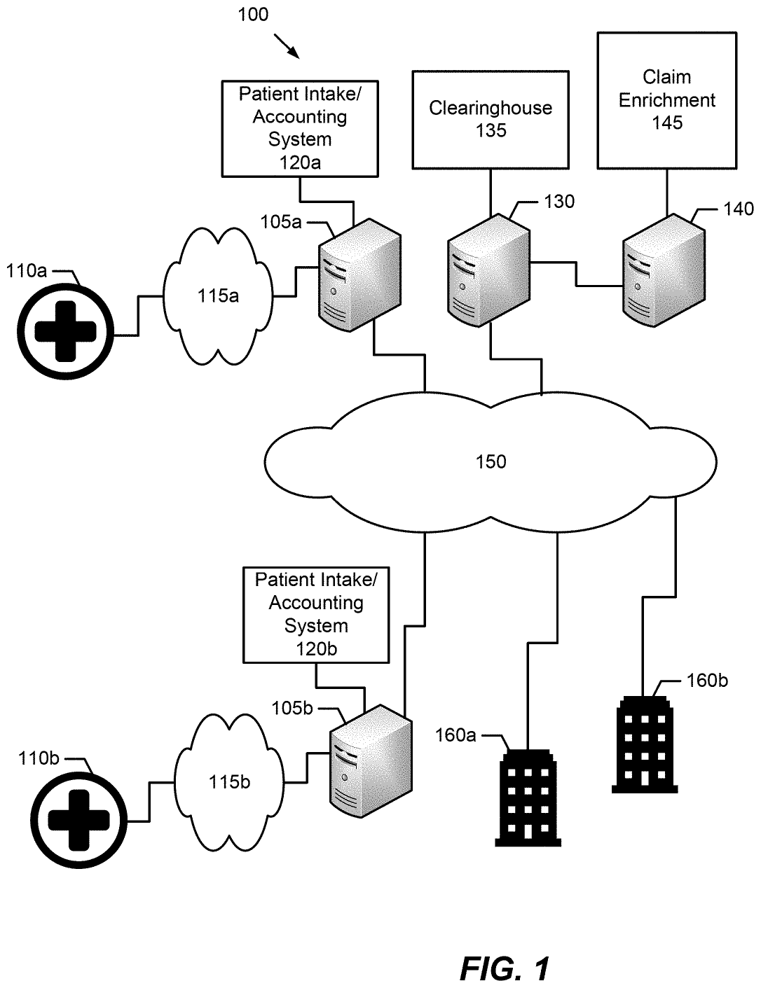

Referring to FIG. 1, a communication network 100 including a clinical enrichment system for supplementing a claim with additional clinical information, in accordance with some embodiments of the inventive concept, comprises multiple health care provider facilities or practices 110a, 110b. Each health care provider facility or practice may represent various types of organizations that are used to deliver health care services to patients via health care professionals, which are referred to generally herein as “providers.” The providers may include, but are not limited to, hospitals, medical practices, mobile patient care facilities, diagnostic centers, lab centers, pharmacies, and the like. The providers may operate by providing health care services for patients and then invoicing one or more payors 160a and 160b for the services rendered. The payors 160a and 160b may include, but are not limited to, providers of private insurance plans, providers of government insurance plans (e.g., Medicare, Medicaid, state, or federal public employee insurance plans), providers of hybrid insurance plans (e.g., Affordable Care Act plans), private of private medical cost sharing plans, and the patients themselves. Two provider facilities 110a, 110b are illustrated in FIG. 1 with the first provider including a first patient intake/accounting system server 105a accessible via a network 115a. The first patient intake/accounting system server 105a may be configured with a patient intake/accounting system module 120a to manage the intake of patients for appointments and to generate invoices for payors for services and products rendered through the provider 110a. The network 115a communicatively couples the first patient intake/accounting system server 105a to other devices, terminals, and systems in the provider's facility 110a. The network 115a may comprise one or more local or wireless networks to communicate with first patient intake/accounting system server 105a when the first patient intake/accounting system server 105a is located in or proximate to the health care service provider facility 110a. When the first patient intake/accounting system server 105a is in a remote location from the health care facility, such as part of a cloud computing system or at a central computing center, then the network 115a may include one or more wide area or global networks, such as the Internet. The second provider facility 110b is similar to the first provider facility 110a and includes a second patient intake/accounting system server 105b, which is configured with a patient intake/accounting system server 120b. The second patient intake/accounting system server 105b is coupled to other devices, terminals, and systems in the provider's facility 110b via a network 115b.

According to embodiments of the inventive concept, an intermediary may be used between a health care service provider and a payor for supplementing claims with additional clinical information when that information may be needed for the payor to efficiently adjudicate the claims. An intermediary server 130 may include a clearinghouse system module 135 that may be configured to receive incoming claims from one or more providers 110a, 110b, supplement these claims, when needed, with additional clinical information, communicate the claims, whether supplemented or not supplemented, to the appropriate payor 160a, 160b, and route the payor responses back to the appropriate provider 110a, 110b by way of the patient intake/accounting systems 120a, 120b. The intermediary may further include a claim processing server 140 that includes a claim enrichment module 145. The claim enrichment module 145 may be configured to determine which claims are likely to need additional clinical information before a payor can fully adjudicate the claim and approve the claim for payment. The claim enrichment module 145 may determine whether additional clinical information may be needed to adjudicate a claim using a variety of techniques including, for example, the application of business rules provided by a payor and/or through use of an AI engine that is configured to learn associations between information contained in a claim and the need to supplement that information with additional clinical information for a payor to adjudicate the claim. The intermediary server 130, the clearinghouse system module 135, the claim processing server 140, and the claim enrichment module 145 may be viewed collectively as a claim enrichment system for supplementing a claim with additional clinical information in accordance with some embodiments of the inventive concept.

A network 150 couples the patient intake/accounting system servers 105a, 105b to the intermediary server 130 and couples the payors 160a and 160b to the eligibility/coverage interface system server 130. The network 150 may be a global network, such as the Internet or other publicly accessible network. Various elements of the network 150 may be interconnected by a wide area network, a local area network, an Intranet, and/or other private network, which may not be accessible by the general public. Thus, the communication network 150 may represent a combination of public and private networks or a virtual private network (VPN). The network 150 may be a wireless network, a wireline network, or may be a combination of both wireless and wireline networks.

The claim enrichment service provided through the intermediary server 130, the clearinghouse system module 135, the claim processing server 140, and the claim enrichment module 145 for supplementing claims with additional clinical information may, in some embodiments, be embodied as a cloud service. For example, health care service providers and/or payors may access the claim enrichment system as a Web service. In some embodiments, the claim enrichment system service may be implemented as a Representational State Transfer Web Service (RESTful Web service).

Although FIG. 1 illustrates an example communication network including a claim enrichment system service for supplementing claims with additional clinical information, it will be understood that embodiments of the inventive subject matter are not limited to such configurations, but are intended to encompass any configuration capable of carrying out the operations described herein.

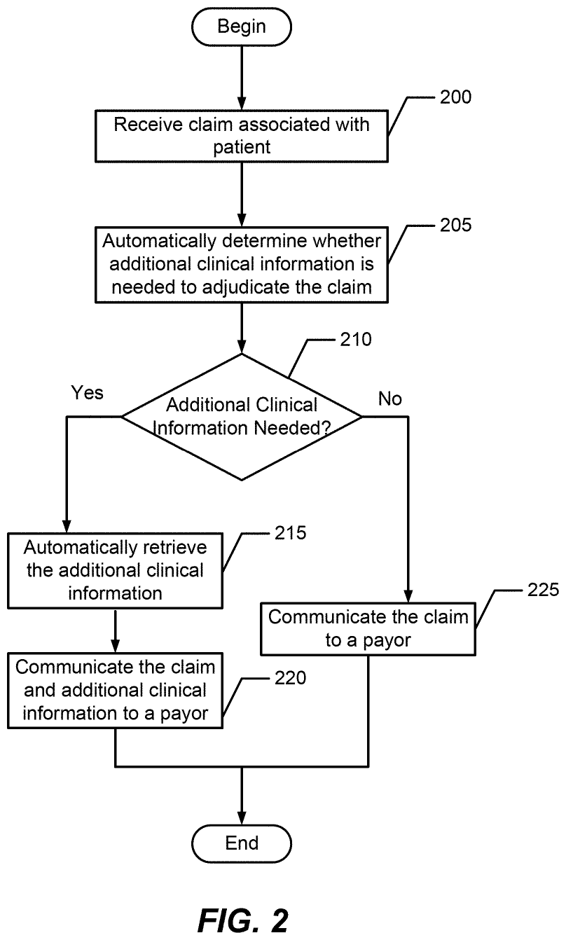

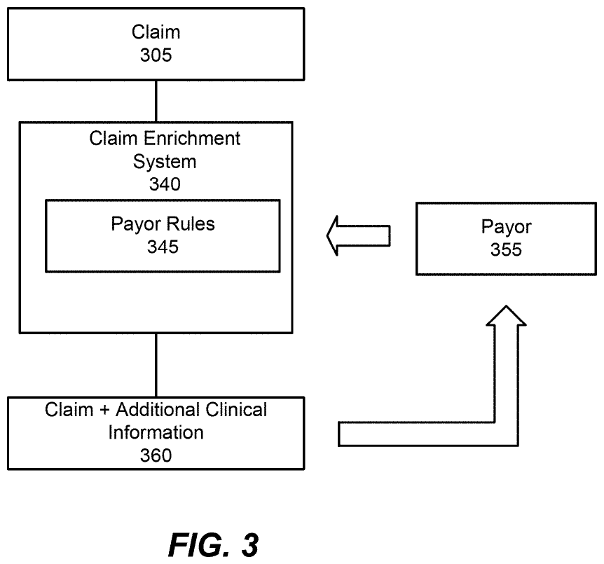

FIG. 2 is a flowchart that illustrates operations for supplementing a claim with additional clinical information in accordance with some embodiments of the inventive concept. Referring now to FIG. 2, operations begin at block 200 where a claim is received at the claim enrichment system from a provider that is associated with a patient or from a clearinghouse/switch that received the claim from a provider. A determination is automatically made at block 205 whether additional clinical information is needed to adjudicate the claim. The determination may be made in multiple ways and may be based on a variety of different factors. FIG. 3 is a block diagram that illustrates embodiments of the claim enrichment system in which payor rules are used to determine whether additional clinical information is needed to adjudicate the claim. Referring to FIG. 3, a claim 305 is provided to the claim enrichment system 340. The claim enrichment system is configured with payor rules 345 that are provided by a payor 355. These payor rules 345 may specify the types of information, including quantitative information, such as thresholds, ranges, and the like, that may be contained in a claim that would result in the payor requiring additional clinical information for adjudicating the claim. The types of information or factors that may be used in making the determination of whether additional clinical information is needed to adjudicate the claim may include the presence of one or more procedure codes (e.g., Current Procedural Terminology (CPT) codes) that are indicative of diagnoses, procedures, services, medications, or other forms of health care services or products. These procedure codes may be indicative of services and/or products that are of questionable medical necessity. Another type of information or factor that may be considered is whether the claim includes information of questionable accuracy. The claim enrichment system 340 may be configured to analyze the claim for internal consistency and may evaluate the particular provider that is submitting the claim based on the payor rules 345. A payor may require providers that have a history of submitting claims with errant or incorrect information to provide supporting clinical information for the services and products listed on the claim more often than providers that have a history of submitting claims with lower error rates. The claim enrichment system 340 may be further configured to take into account the risk adjustment factor associated with the patient based on the payor rules 345. A payor may require additional clinical information for a claim that lists services and/or products that are inconsistent with the expected healthcare use for the patient based on the patient's risk adjustment factor. Based on application of the payor rules, a determination may be made whether additional clinical information is needed to adjudicate the claim and whether the claim includes this additional clinical information in the form of an attachment, addendum, or the like. The claim may be supplemented with additional clinical information 360, if needed, and may be communicated to the payor 355 for adjudication.

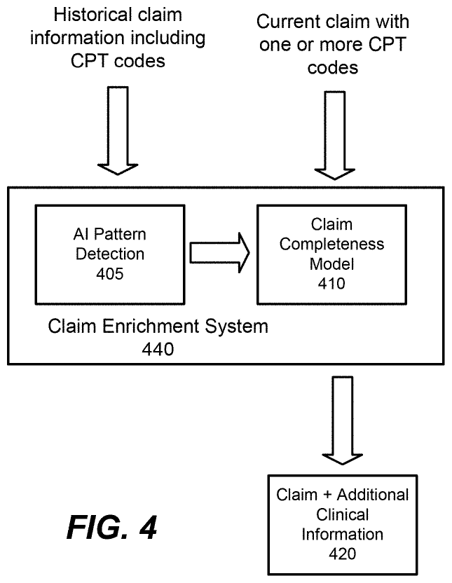

In accordance with other embodiments of the inventive concept, the claim enrichment system may use an AI engine in addition to or instead of the payor rules 345 to determine whether a claim is to be supplemented with additional clinical information. FIG. 4 is a block diagram that illustrates embodiments of the claim enrichment system in which an AI engine is used to determine whether a claim is to be supplemented with additional clinical information to facilitate adjudication by a payor. As shown in FIG. 4, the claim enrichment system 440 includes an AI engine, which may be a machine learning engine comprising an AI pattern detection module 405 and a claim completeness model 410. The AI pattern detection module 405 is configured to receive historical claim information, which may include, but is not limited to, CPT codes, provider names, patient risk adjustment factors, and claim accuracy errors, and may learn associations between the historical claim information and instances in which the claims were supplemented with additional clinical information. The AI pattern detection module 405 may then generate a claim completeness model 410 based on these learned associations, which can be used to process a current claim, which may include one or more CPT codes along with additional information, such as provider name, patient risk adjustment factor, etc. to determine whether the claim should be supplemented with additional clinical information. The claim plus additional clinical information 420 may be output for communication to a payor.

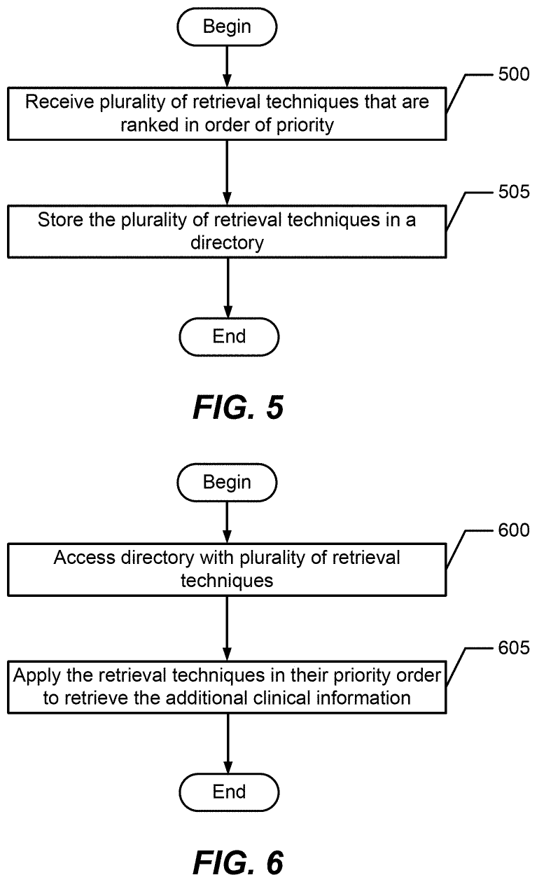

Returning to FIG. 2, the determination is made at block 210 whether additional clinical information is needed for the payor to adjudicate the claim. If additional clinical information is needed, then the additional clinical information is automatically retrieved at block 215. Embodiments for automatically retrieving the additional clinical information will now be described with respect to the flowcharts of FIGS. 5 and 6. In some embodiments, a directory may be maintained in which one or more retrieval techniques are stored. In some embodiments, when the directory includes a plurality of retrieval techniques, the retrieval techniques may be stored in priority order. Thus, at block 500, one or more retrieval techniques may be received from a provider. In some embodiments, when multiple retrieval techniques are received, the retrieval techniques may be ranked in order of priority. These retrieval techniques may then be stored in a directory at block 505. In general, the retrieval techniques may comprise one or more automated data pull techniques and/or one or more automated request-response techniques. In accordance with some embodiments of the inventive concept, the retrieval techniques may include, but are not limited to, accessing a storage location to retrieve the additional clinical information using Fast Healthcare Interoperability Resources (FHIR) protocol, sending a request to the provider to return the additional clinical information in an Electronic Data Interchange (EDI) format, or sending an electronic mail message to the provider requesting the additional clinical information. As described above, the storage area for the clinical information, according to various embodiments of the inventive concept, may be implemented in a variety of different ways. In some embodiments, the storage area may be part of one or more clinical networks that may be used to implement a local area, wide area, or national health information exchange. In other embodiments, a provider may implement its own network endpoint for housing clinical data and the provider may grant other entities direct access to the electronic health records stored at this end point. Referring now to FIG. 6, the claim enrichment system may access the directory containing the one or more retrieval techniques at block 600. The retrieval techniques may then be applied at block 605 in priority order until all of the additional clinical information that will accompany the claim is retrieved. Multiple retrieval techniques may be used to retrieve the additional clinical information as not all of the clinical information may be accessible via the same retrieval technique. Even in circumstances where all of the information may be accessible via the same retrieval technique, there may be portions of the information that is accessible via a faster retrieval technique so that it may be more efficient to use the faster retrieval technique for retrieving portion of the information while using a slower retrieval technique to retrieve the remainder of the information.

Returning to FIG. 2, the claim plus the additional clinical information is communicated to the payor at block 220. If the determination at block 210 is that additional clinical information is not needed to supplement the claim, then the claim without any supplementation is communicated to the payor at block 225.

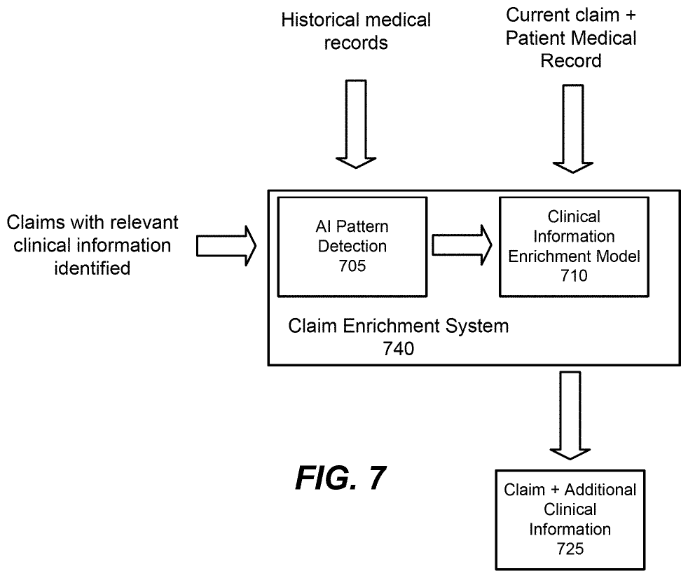

The additional clinical information that may supplement a claim is often in the form of a patient's medical record. Patient medical records, however, are often large and may include extra information that may not be used by a payor in adjudicating the claim. In some embodiments of the inventive concept, the claim enrichment system may use an AI engine to extract a portion of a patient's medical record that is relevant to adjudication of a claim allowing the rest of the patient's medical record to be discarded. The claim may then be supplemented with only the extracted portion of the medical record for adjudication by the payor. FIG. 7 is a block diagram that illustrates embodiments of the claim enrichment system in which an AI engine is used to extract a portion of a patient's medical record to supplement a claim for processing by a payor. As shown in FIG. 7, the claim enrichment system 740 includes an AI pattern detection module 705 and a clinical information enrichment model 710. The AI pattern detection module 705 is configured to receive historical medical records for patients along with patient claims with the relevant clinical information identified and may learn associations between the information contained in the historical medical records and the particular relevant clinical information that has been used to supplement the claims. The AI pattern detection module 705 may then generate a clinical information enrichment model 710 based on these learned associations, e.g., associations between procedure codes, provider names, and/or patient risk adjustment factors in the historical claims and the patient medical record information that has historically been used to supplement claims. The clinical information enrichment model 710 may be used to process a current claim with an associated patient medical record to extract a portion of the patient's medical record to be included as supplemental clinical information for the payor. The claim plus the additional clinical information extracted from the patient's medical record 725 may be communicated to the payor.

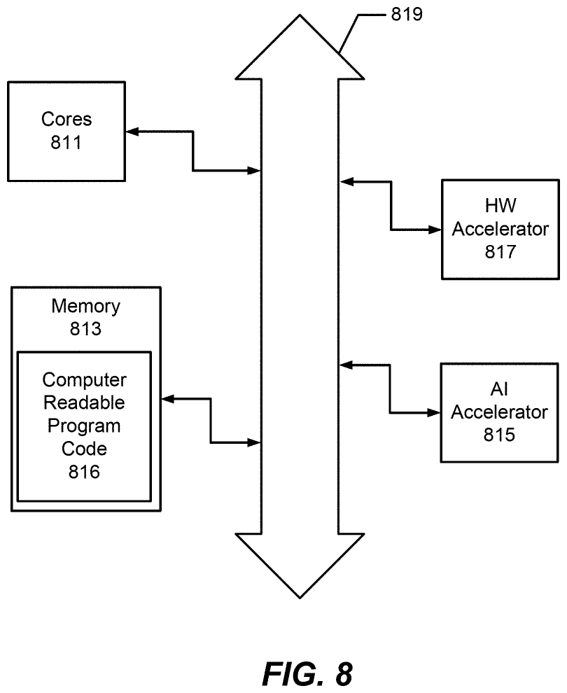

FIG. 8 is a block diagram of a data processing system that may be used to implement the claim processing server 140 of FIG. 1 and/or the claim enrichment systems 340, 440, and 740 of FIGS. 3, 4, and 7 in accordance with some embodiments of the inventive concept. As shown in FIG. 8, the data processing system may include at least one core 811, a memory 813, an artificial intelligence (AI) accelerator 815, and a hardware (HW) accelerator 817. The at least one core 811, the memory 813, the AI accelerator 815, and the HW accelerator 817 may communicate with each other through a bus 819.

The at least one core 811 may be configured to execute computer program instructions. For example, the at least one core 811 may execute an operating system and/or applications represented by the computer readable program code 816 stored in the memory 813. In some embodiments, the at least one core 811 may be configured to instruct the AI accelerator 815 and/or the HW accelerator 817 to perform operations by executing the instructions and obtain results of the operations from the AI accelerator 815 and/or the HW accelerator 817. In some embodiments, the at least one core 811 may be an ASIP customized for specific purposes and support a dedicated instruction set.

The memory 813 may have an arbitrary structure configured to store data. For example, the memory 813 may include a volatile memory device, such as dynamic random-access memory (DRAM) and static RAM (SRAM), or include a non-volatile memory device, such as flash memory and resistive RAM (RRAM). The at least one core 811, the AI accelerator 815, and the HW accelerator 817 may store data in the memory 813 or read data from the memory 813 through the bus 819.

The AI accelerator 815 may refer to hardware designed for AI applications. In some embodiments, the AI accelerator 815 may include a machine learning engine configured to determine whether additional clinical information is needed to adjudicate a claim and/or to identify relevant portions of a patient's medical record to include with the claim as supplemental clinical information. The AI accelerator 815 may generate output data by processing input data provided from the at least one core 815 and/or the HW accelerator 817 and provide the output data to the at least one core 811 and/or the HW accelerator 817. In some embodiments, the AI accelerator 815 may be programmable and be programmed by the at least one core 811 and/or the HW accelerator 817. The HW accelerator 817 may include hardware designed to perform specific operations at high speed. The HW accelerator 817 may be programmable and be programmed by the at least one core 811.

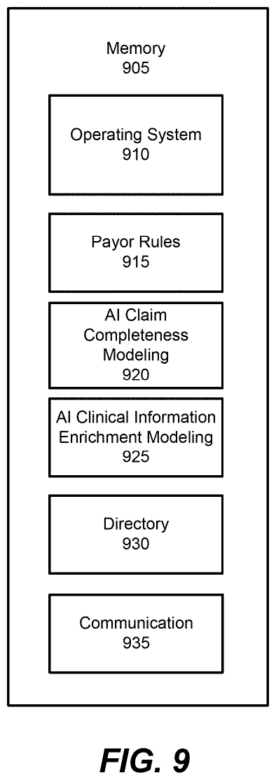

FIG. 9 illustrates a memory 905 that may be used in embodiments of data processing systems, such as the claim processing server 140 of FIG. 1, the claim enrichment systems 340, 440, and 740 of FIGS. 3, 4, and 7, and the data processing system of FIG. 7, respectively, to facilitate supplementing a claim with additional clinical information. The memory 905 is representative of the one or more memory devices containing the software and data used for facilitating operations of the claims processing server 140 and the claim enrichment module 145 as described herein. The memory 905 may include, but is not limited to, the following types of devices: cache, ROM, PROM, EPROM, EEPROM, flash, SRAM, and DRAM. As shown in FIG. 9, the memory 905 may contain seven or more categories of software and/or data: an operating system 910, payor rules 915, an AI claim completeness modeling module 920, an AI clinical information enrichment modeling module 925, a directory 930, and a communication module 935. In particular, the operating system 810 may manage the data processing system's software and/or hardware resources and may coordinate execution of programs by the processor.

The payor rules module 915 may provide the payor rules 345 for determining whether additional clinical information is needed for adjudication of a claim as described above with respect to FIG. 3. The AI completeness modeling module 920 may be configured to perform one or more of the operations described above with respect to the claim enrichment system 440 of FIG. 4. The AI clinical information enrichment modeling module 925 may be configured to perform one or more of the operations described above with respect to the claim enrichment system 740 of FIG. 7. The directory 930 may provide the directory for storage of the various retrieval techniques that may be used to obtain clinical information from a provider as described with respect to FIGS. 5 and 6. The communication module 935 may be configured to facilitate communication between the claim processing server 140 of FIG. 1 and/or the claim enrichment systems 340, 440, and 740 of FIGS. 3, 4, and 7 and the providers 110a, 110b and payors 160a, 160b of FIG. 1.

Although FIGS. 8 and 9 illustrate hardware/software architectures that may be used in data processing systems, such as the claim processing server 140 of FIG. 1, the claim enrichment systems 340, 440, and 740 of FIGS. 3, 4, and 7, and the data processing system of FIG. 8, respectively, in accordance with some embodiments of the inventive concept, it will be understood that the present invention is not limited to such a configuration but is intended to encompass any configuration capable of carrying out operations described herein.

Computer program code for carrying out operations of data processing systems discussed above with respect to FIGS. 1-8 may be written in a high-level programming language, such as Python, Java, C, and/or C++, for development convenience. In addition, computer program code for carrying out operations of the present invention may also be written in other programming languages, such as, but not limited to, interpreted languages. Some modules or routines may be written in assembly language or even micro-code to enhance performance and/or memory usage. It will be further appreciated that the functionality of any or all of the program modules may also be implemented using discrete hardware components, one or more application specific integrated circuits (ASICs), or a programmed digital signal processor or microcontroller.

Moreover, the functionality of the intermediary server 130 of FIG. 1, the claim processing server 140 of FIG. 1, the claim enrichment systems 340, 440, and 740 of FIGS. 3, 4, and 7, and the data processing system of FIG. 8 may each be implemented as a single processor system, a multi-processor system, a multi-core processor system, or even a network of stand-alone computer systems, in accordance with various embodiments of the inventive concept. Each of these processor/computer systems may be referred to as a “processor” or “data processing system.” The functionality provided by the intermediary server 130 and the claim processing server 140 may be merged into a single server or maintained as separate servers in accordance with different embodiments of the inventive concept.

The data processing apparatus described herein with respect to FIGS. 1-8 may be used to facilitate supplementing a claim with additional clinical information according to some embodiments of the inventive concept described herein. These apparatus may be embodied as one or more enterprise, application, personal, pervasive and/or embedded computer systems and/or apparatus that are operable to receive, transmit, process and store data using any suitable combination of software, firmware and/or hardware and that may be standalone or interconnected by any public and/or private, real and/or virtual, wired and/or wireless network including all or a portion of the global communication network known as the Internet, and may include various types of tangible, non-transitory computer readable media. In particular, the memory 905 when coupled to a processor includes computer readable program code that, when executed by the processor, causes the processor to perform operations including one or more of the operations described herein with respect to FIGS. 1-7.

Some embodiments of the inventive concept may provide a claim enrichment system that can improve efficiency and reduce costs for payors when adjudicating claims from providers by eliminating manual tasks performed to obtain clinical information from providers to supplement claims. This may allow payors to review more clinical data when adjudicating claims due to the lower cost barriers involved in supplementing claims with clinical information from patient medical records. This may result in improved payment accuracy, improved risk adjustment for insured patient groups, improved medical cost management, and/or improved care coordination for customers of a payor, e.g., insured patients.

### Further Definitions and Embodiments

In the above-description of various embodiments of the present inventive concept, it is to be understood that the terminology used herein is for the purpose of describing particular embodiments only and is not intended to be limiting of the invention. Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of this specification and the relevant art and will not be interpreted in an idealized or overly formal sense expressly so defined herein.

The flowchart and block diagrams in the figures illustrate the architecture, functionality, and operation of possible implementations of systems, methods, and computer program products according to various aspects of the present inventive concept. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular aspects only and is not intended to be limiting of the inventive concept. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Like reference numbers signify like elements throughout the description of the figures.

In the above-description of various embodiments of the present inventive concept, aspects of the present inventive concept may be illustrated and described herein in any of a number of patentable classes or contexts including any new and useful process, machine, manufacture, or composition of matter, or any new and useful improvement thereof. Accordingly, aspects of the present inventive concept may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present inventive concept may take the form of a computer program product comprising one or more computer readable media having computer readable program code embodied thereon.

Any combination of one or more computer readable media may be used. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

The description of the present inventive concept has been presented for purposes of illustration and description, but is not intended to be exhaustive or limited to the inventive concept in the form disclosed. Many modifications and variations will be apparent to those of ordinary skill in the art without departing from the scope and spirit of the inventive concept. The aspects of the inventive concept herein were chosen and described to best explain the principles of the inventive concept and the practical application, and to enable others of ordinary skill in the art to understand the inventive concept with various modifications as are suited to the particular use contemplated.

What is claimed is:

1. A method, comprising:
receiving a claim associated with a patient from a provider;
automatically determining whether additional clinical information is needed to adjudicate the claim;
automatically retrieving the additional clinical information responsive to determining that the additional clinical information is needed to adjudicate the claim;
communicating the claim and the additional clinical information to a payor responsive to determining that the additional clinical information is needed to adjudicate the claim; and
communicating the claim to the payor responsive to determining that the additional clinical information is not needed to adjudicate the claim.

2. The method of claim 1, wherein determining whether the additional clinical information is needed to adjudicate the claim comprises:
determining that additional clinical information is needed to adjudicate the claim when the claim includes one or more procedure codes known to require the additional clinical information, the claim includes one or more procedure codes indicative of a health care procedure of questionable medical necessity, the claim includes information of questionable accuracy, or the claim includes one or more procedure codes that are inconsistent with a risk adjustment factor associated with the patient.

3. The method of claim 1, wherein automatically retrieving the additional clinical information comprises:
accessing a directory that includes a plurality of retrieval techniques for retrieving the additional clinical information that are ranked in order of priority; and
applying respective ones of the plurality of retrieval techniques in the order of priority to retrieve the additional clinical information.

4. The method of claim 3, further comprising:
receiving the plurality of retrieval techniques that are ranked in order of priority from the provider; and
storing the plurality of retrieval techniques in the directory.

5. The method of claim 3, wherein the plurality retrieval techniques comprises an automated data pull technique and a request-response technique.

6. The method of claim 3, wherein the plurality of retrieval techniques comprises accessing a storage location to retrieve the additional clinical information using Fast Healthcare Interoperability Resources (FHIR) protocol, sending a request to the provider to return the additional clinical information in an Electronic Data Interchange (EDI) format, or sending an electronic mail message to the provider requesting the additional clinical information.

7. The method of claim 1, wherein automatically determining whether additional clinical information is needed to adjudicate the claim comprises:
generating a claim completeness model based on adjudication of historical claims in which associations are determined between procedure codes, provider names, and/or patient risk adjustment factors in the historical claims and a need for additional clinical information.

8. The method of claim 7, wherein generating the claim completeness model comprises:
using an Artificial Intelligence (AI) system to learn the associations between the procedure codes, the provider names, and/or the patient risk adjustment factors in the historical claims and the need for additional clinical information.

9. The method of claim 1, wherein automatically retrieving the additional clinical information comprises:
retrieving a medical record associated with the patient; and
processing the medical record to extract the additional clinical information therefrom.

10. The method of claim 9, wherein processing the medical record to extract the additional clinical information therefrom comprises:
generating a clinical information enrichment model based on supplementation of historical claims with patient medical record information.

11. The method of claim 10, wherein generating the clinical information enrichment model comprises:
using an Artificial Intelligence (AI) system to learn associations between procedure codes, provider names, and/or patient risk adjustment factors in the historical claims and the patient medical record information.

12. A system, comprising:
a processor; and
a memory coupled to the processor and comprising computer readable program code embodied in the memory that is executable by the processor to perform operations comprising:
receiving a claim associated with a patient from a provider;
automatically determining whether additional clinical information is needed to adjudicate the claim;
automatically retrieving the additional clinical information responsive to determining that the additional clinical information is needed to adjudicate the claim;
communicating the claim and the additional clinical information to a payor responsive to determining that the additional clinical information is needed to adjudicate the claim; and
communicating the claim to the payor responsive to determining that the additional clinical information is not needed to adjudicate the claim.

13. The system of claim 12, wherein determining whether the additional clinical information is needed to adjudicate the claim comprises:
determining that additional clinical information is needed to adjudicate the claim when the claim includes one or more procedure codes known to require the additional clinical information, the claim includes one or more procedure codes indicative of a health care procedure of questionable medical necessity, the claim includes information of questionable accuracy, or the claim includes one or more procedure codes that are inconsistent with a risk adjustment factor associated with the patient.

14. The system of claim 12, wherein automatically retrieving the additional clinical information comprises:
accessing a directory that includes a plurality of retrieval techniques for retrieving the additional clinical information that are ranked in order of priority; and
applying respective ones of the plurality of retrieval techniques in the order of priority to retrieve the additional clinical information.

15. The system of claim 12, wherein automatically determining whether additional clinical information is needed to adjudicate the claim comprises:
generating a claim completeness model based on adjudication of historical claims in which associations are determined between procedure codes in the historical claims and a need for additional clinical information.

16. The system of claim 15, wherein generating the claim completeness model comprises:
using an Artificial Intelligence (AI) system to learn the associations between the procedure codes in the historical claims and the need for additional clinical information.

17. The system of claim 12, wherein automatically retrieving the additional clinical information comprises:
retrieving a medical record associated with the patient; and
processing the medical record to extract the additional clinical information therefrom.

18. The system of claim 17, wherein processing the medical record to extract the additional clinical information therefrom comprises:
generating a clinical information enrichment model based on supplementation of historical claims with patient medical record information.

19. The system of claim 18, wherein generating the clinical information enrichment model comprises:
using an Artificial Intelligence (AI) system to learn associations between procedure codes in the historical claims and the patient medical record information.

20. A computer program product, comprising:
a non-transitory computer readable storage medium comprising computer readable program code embodied in the medium that is executable by a processor to perform operations comprising:
receiving a claim associated with a patient from a provider;
automatically determining whether additional clinical information is needed to adjudicate the claim;
automatically retrieving the additional clinical information responsive to determining that the additional clinical information is needed to adjudicate the claim;
communicating the claim and the additional clinical information to a payor responsive to determining that the additional clinical information is needed to adjudicate the claim; and
communicating the claim to the payor responsive to determining that the additional clinical information is not needed to adjudicate the claim.
